SEQUENCE LISTING (3 sequences)

SEQ ID NO: 1 - protein, 13 aa, synthetic construct
  source 1..13: note = PADRE peptide
AKFVAAWTLKAAA

SEQ ID NO: 2 - protein, 13 aa, synthetic construct
  source 1..13: note = PADRE peptide
  SITE 3: note = cyclohexylalanine
AKXVAAWTLKAAA

SEQ ID NO: 3 - protein, 23 aa, synthetic construct
  source 1..23: note = PADRE-E10
  SITE 1: note = D-alanine
  SITE 3: note = cyclohexylalanine
  SITE 13: note = D-alanine
AKXVAAWTLKAAAEEEEEEEEEE

(19) United States
(12) Patent Application Publication
(10) Pub. No.: US 2025/0032607 A1
(43) Pub. Date: Jan. 30, 2025

(54) LIPID NANOPARTICLE-BASED ANTI-FENTANYL VACCINE

(71) Applicant: Universiteit Gent, Gent (BE)

(72) Inventors: Bruno De Geest, Ghent (BE); Zifu Zhong, Ghent (BE); Yong Chen, Ghent (BE); Yi HUANG, Ghent (BE); Tingting YE, Ghent (BE)

(73) Assignee: Universiteit Gent, Gent (BE)

(21) Appl. No.: 18/767,360

(22) Filed: Jul. 9, 2024

Related U.S. Application Data
(60) Provisional application No. 63/525,760, filed on Jul. 10, 2023.

(51) Int. Cl. A61K 39/385; A61K 9/51; A61K 39/00; A61K 47/60; A61K 47/69; A61P 37/04

(52) U.S. Cl. CPC A61K 39/385; A61K 9/5123; A61K 39/0013; A61K 47/60; A61K 47/6925; A61P 37/04; A61K 2039/55516

(57) ABSTRACT

The present invention provides a lipid nanoparticle comprising a fentanyl hapten, and a T helper peptide and/or an adjuvant, wherein the fentanyl hapten is conjugated to the outer surface of the lipid nanoparticle, and wherein the T helper peptide and/or the adjuvant is/are encapsulated within the lipid nanoparticle. The invention further provides a pharmaceutical composition comprising the lipid nanoparticle and uses thereof for inducing an immune response against fentanyl and the prevention or treatment of a fentanyl abuse disorder or a fentanyl addiction or a fentanyl overdose in a subject. Further provided herein is a method for preparing the lipid nanoparticle of the invention.

### CROSS-REFERENCE TO RELATED APPLICATIONS

This application claims the benefit of U.S. Provisional Patent Application No. 63/525,760, filed Jul. 10, 2023, the contents of which are incorporated herein by reference in their entirety.

### INCORPORATION BY REFERENCE OF SEQUENCE LISTING XML

The Sequence Listing entitled “20230705 UG-111-US-PROV SEQLIST.xml”, 5 KB, created on Jul. 5, 2023, submitted electronically using the U.S. Patent Center, is incorporated by reference as the Sequence Listing XML for the subject application.

### FIELD

The present application broadly relates to the field of vaccine technology, and in particular relates to anti-fentanyl vaccines. More particularly, the present application provides lipid nanoparticles for an anti-fentanyl vaccine, a pharmaceutical composition, including a vaccine composition, comprising the lipid nanoparticles, uses thereof for administration to a subject in need thereof and methods for preparing the same.

### BACKGROUND

The opioid use disorder epidemic is ravaging the western society, with high rates of relapse and overdose deaths in for example the United States and Europe. The US witnessed a high number of opioid-related deaths, with a subsequent 100% increase in fatal overdoses involving synthetic opioids from 2015 to 2016. Illicitly manufactured fentanyl and its analogs contribute to over 50% of opioid-related fatalities. Fentanyl, a schedule II opioid agonist, surpasses morphine in potency by 100-200 times, making it a popular adulterant in heroin and counterfeit prescription opioids due to its easy synthesis and low cost.

Anti-opioid vaccines hold great potential to reduce long-term opioid use and the risk of accidental overdose when used in combination with other treatments against addiction. At present, there are no vaccines available on the market for the treatment of substance use disorders.

Current anti-opioid vaccines in pre-clinical development focus on bio-conjugates composed of opioid haptens that are chemically bound to an immunogenic carrier protein in combination with an admixed molecular adjuvant. This approach is sub-optimal on multiple levels. First, this approach is not designed to co-deliver the hapten-protein conjugate with the molecular adjuvant to antigen presenting cells in lymphoid tissue. Furthermore, the arbitrary conjugation of haptens to a carrier poses challenges in terms of reproducibility, thereby impacting manufacturability. Additionally, this approach exposes the hapten motifs to the immune system in a suboptimal manner.

There thus remains a need for improved anti-fentanyl vaccines.

### SUMMARY

The current invention is based on an innovative lipid nanoparticle (LNP) that encapsulates a fentanyl hapten together with an immunogen and/or a molecular adjuvant in a single LNP. Advantageously, the LNP offers the flexibility to select one or more molecular adjuvants for encapsulation within the LNP.

As shown in the experimental section, the LNPs described herein induced fentanyl-specific immune responses, producing antibodies with great affinity. Without wishing to be bound by any theory, the exposure of the hapten motifs in a spherical conformation, similar as the protein motifs on a viral particle surface, may yield vastly improved binding to B cell receptors and hence may induce anti-fentanyl antibodies with vastly increased affinity. Also, the design of the LNP allows the co-delivery of hapten, immunogen and/or molecular adjuvant to antigen presenting cells in lymphoid tissue, thereby yielding a vastly improved immune response.

As shown also in the experimental section, the immune sera could potently reduce mu opioid receptor activation. Therefore, pharmaceutical compositions, in particular vaccines, which include these novel LNPs, hold potential to alleviate toxic effects of fentanyl exposure, thereby reducing the psychoactive impact of fentanyl class drugs. As such, the present invention can address the urgent need to combat the fentanyl crisis by safeguarding recreational drug users and individuals in high-risk professions from accidental exposure and its potentially lethal consequences. The implementation of LNPs according to the invention and pharmaceutical compositions comprising these LNPs have the potential to save lives, decrease fatal overdoses, and mitigate the impact of fentanyl on affected communities.

In view of these advantages, an aspect of the invention provides a lipid nanoparticle comprising a fentanyl hapten, and a T helper peptide and/or an adjuvant, wherein the fentanyl hapten is conjugated to the outer surface of the lipid nanoparticle, and wherein the T helper peptide and/or the adjuvant is/are encapsulated within the lipid nanoparticle.

Another aspect provides a pharmaceutical composition comprising a lipid nanoparticle of the invention and a pharmaceutically acceptable carrier.

A further aspect provides a lipid nanoparticle or a pharmaceutical composition of the invention for use in medicine.

An aspect provides a method for inducing an immune response against fentanyl in a subject, said method comprising administering to the subject an immunologically effective amount of the lipid nanoparticle or the pharmaceutical composition as described herein.

Another aspect provides a method for the prevention or treatment of a fentanyl abuse disorder, a fentanyl addiction or a fentanyl overdose in a subject, said method comprising administering a therapeutically effective amount of the lipid nanoparticle or the pharmaceutical composition of the invention to the subject.

A further aspect relates to a method for preparing a lipid nanoparticle of the invention comprising:
mixing an aqueous composition comprising the T helper peptide conjugated to an anionic peptide or polypeptide with a lipid composition comprising a cationic ionizable lipid, the fentanyl hapten conjugated to a lipid via a hydrophilic, non-immunogenic polymer, preferably the fentanyl hapten conjugated to a PEG-modified lipid, a structural helper lipid, and/or a lipid-conjugated adjuvant in a suitable organic solvent, such as ethanol, to form a mixture; and
removing the organic solvent from the mixture, thereby obtaining the lipid nanoparticle.

These and further aspects and preferred embodiments of the invention are described in the following sections and in the appended claims. The subject-matter of the appended claims is hereby specifically incorporated in this specification.

### BRIEF DESCRIPTION OF DRAWINGS

The following description of the figures of specific embodiments of the invention is merely exemplary in nature and is not intended to limit the present teachings, their application or uses.

FIG. 1. Schematic representation of anti-fentanyl LNP-based nanovaccine technology.

FIG. 2. Mass-spectrometry analysis of compound 5 in the lipid-PEG fentanyl hapten synthesis scheme shown in example 1.

FIG. 3. Post-prime ELISA dilution curves from mice immunized with a pharmaceutical composition comprising a fentanyl LNP according to an embodiment of the invention and controls.

FIG. 4. Fentanyl-specific antibody titers from mice immunized with a pharmaceutical composition comprising a fentanyl LNP according to an embodiment of the invention and controls.

FIG. 5. In vitro inhibition of mu opioid receptor activation by sera from mice immunized with LNPs according to embodiments of the invention. Ratio of the luminescence recorded from fentanyl-treated cells to the luminescence recorded from morphine-treated cells is shown.

FIG. 6A-6C. (A) Experimental scheme for the analysis of the antinociceptive effects of fentanyl. Balb/c mice received a single i.m. immunization of different fentanyl-containing LNP formulations or PBS (control) and, two-weeks post-immunization, were challenged with a thermal stimulus in the context of escalating doses of fentanyl (n=7, mean±SD). (B) Quantification of fentanyl-induced antinociception as measured by the latency in reacting to a heat stimulus. The latency in response is plotted as % of the maximum possible effect (MPE). (C) ED50 values represent the concentration of fentanyl at 50% of the MPE (n=7, mean±SD). Statistical analysis by one-way ANOVA: *:p>0.5, *:p>0.1 **:p>0.01, ****:p>0,0001).

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements, or method steps. The terms also encompass “consisting of” and “consisting essentially of”.

The recitation of numerical ranges by endpoints includes all integer numbers and, where appropriate, fractions subsumed within the respective ranges, as well as the recited endpoints. This applies to numerical ranges irrespective of whether they are introduced by the expression “from . . . to . . . ” or the expression “between . . . and . . . ” or another expression. Any numerical range recited herein is intended to include all sub-ranges subsumed therein.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the term “one or more”, such as one or more members of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

As used herein, the term “and/or” when used in a list of two or more items, means that any one of the listed items can be employed by itself or any combination of two or more of the listed items can be employed. For example, if a list is described as comprising group A, B, and/or C, the list can comprise A alone, B alone, C alone, A and B in combination, A and C in combination, B and C in combination, or A, B, and C in combination.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

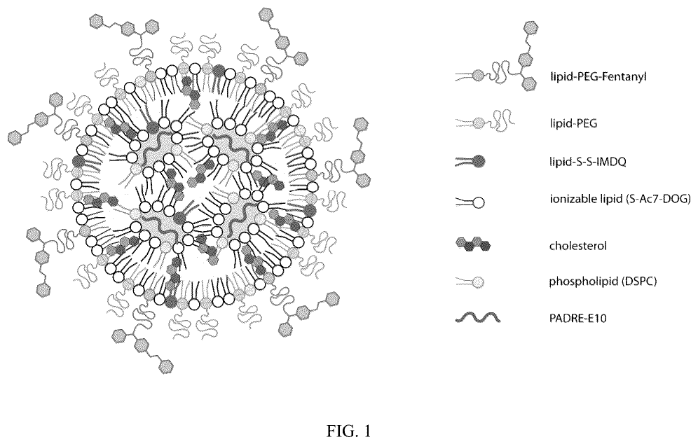

In an aspect, the present invention provides a lipid nanoparticle comprising a fentanyl hapten, and a T helper peptide and/or an adjuvant, wherein the fentanyl hapten is conjugated to the outer surface of the lipid nanoparticle, and wherein the T helper peptide and/or the adjuvant is/are encapsulated within the lipid nanoparticle, such as represented in FIG. 1.

As used herein, a “lipid nanoparticle” (also referred to herein as LNP) refers to a nanosized particle composed of a combination of different lipids. Many different types of lipids may be included in such LNP, including, without limitation ionizable lipids, phospholipids (especially compounds having a phosphatidylcholine group), sterols (e.g. cholesterol), lipids modified with a hydrophilic polymer as described herein such as polyethylene glycol (PEG)-modified lipids. It is understood that the lipid nanoparticle formulation may be influenced by, but not limited to, the selection of the different lipid components, the degree of lipid saturation, the nature of the PEGylation, and the like.

The term “encapsulate” as used herein means to enclose within the lipid nanoparticle, preferably enclose completely within the lipid nanoparticle.

The term “hapten” as used herein refers to small molecules and modified versions thereof which are used as antigens. Haptens are able to act as recognition sites for the production of specific antibodies but cannot by themselves stimulate the necessary immune response. Haptens can be made immunogenic e.g. by coupling them to a suitable carrier, such as a protein or a nanoparticle, which can be processed by antigen presenting cells and presented to the immune system such that the immune system recognizes the unmodified small molecule. Further, the hapten may be characterized as the specificity-determining portion of a hapten-carrier conjugate that is capable of reacting with an antibody specific to the hapten in its free state.

The term “fentanyl” as used herein refers to fentanyl or a fentanyl derivative or both; that is, as further described herein, a fentanyl hapten as used herein may be derived from fentanyl or from a fentanyl derivative. As used herein, a “fentanyl derivative” (also referred to herein as a “derivative of fentanyl” or a “fentanyl analog”) includes analogs of fentanyl such as acetylfentanyl, alfentanil, brifentail, carfentanil, lofentanil, mefentanyl, α-mefentanyl, mirfenantil, ohmefentanyl, phenaridine, remifentanil, sufentanil, trefentanil, etc.

Non-limiting examples of fentanyl haptens that can be used in the LNPs according to the invention include a fentanyl hapten having a structure of any one of formulas (1) to (IV), wherein R is (Gly)₄-OH.

In particular embodiments, the fentanyl hapten has the structure of formula (1) and can also be found as compound 5 in example 1.

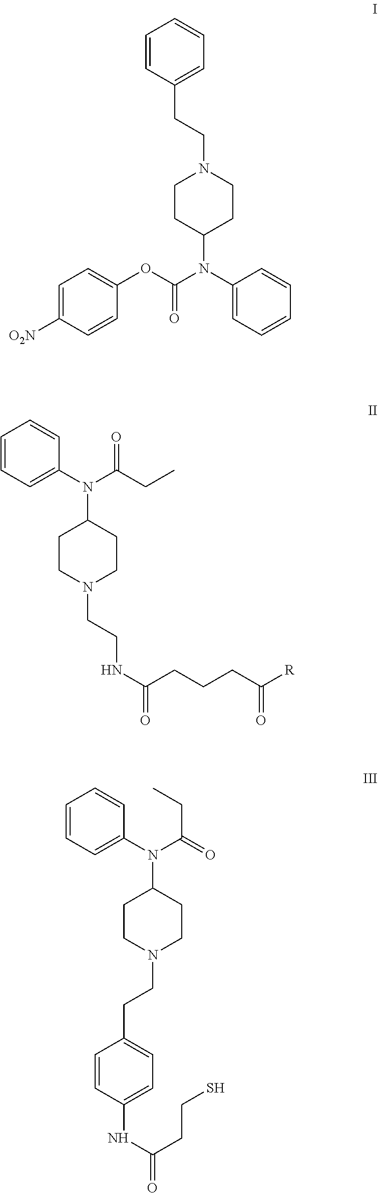

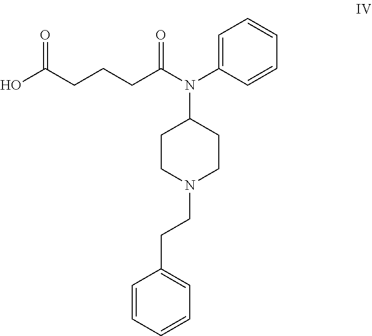 [SMILES: O=C(O)CCCC(=O)N(c1ccccc1)C1CCN(CCc2ccccc2)CC1]

As used herein, the term “T helper peptide” refers to any peptide that is capable of activating a CD4+ T cell, which can in turn provide the necessary help to induce the activation of both CD8+ T cells and B cells. Preferably, the T helper peptide is a universal T helper peptide. As used herein, the term “universal T helper peptide” refers to a T helper peptide that binds to a broad range of MHC class 11 haplotypes, and can thereby activate CD4+ T cells expressing those MHCs. Non-limiting examples of universal T helper peptides include a PADRE, tetanus toxoid or a peptide derived from tetanus toxoid (e.g. as described in Falugi et al. (2001) Eur J Immunol 31: 3816-3824), diphtheria toxin or a peptide derived from diphtheria toxin (e.g. comprising CRM 197, or the tetanus toxin as described in Diethelm-Okita, et al. (2000) J Infec Dis 181: 1001-1009), CD4+ T cell epitope of cholera toxin, a HBV peptide (e.g. HbsAg), measles peptide, or any combination thereof.

In preferred embodiments, the T helper peptide is a pan HLA DR-binding epitope (PADRE), such as the PADRE comprising or consisting of the sequence set forth in SEQ ID NO: 1 (AKFVAAWTLKAAA) or SEQ ID NO: 2 (AKXVAAWTLKAAA), wherein X=cyclohexylalanine.

In certain embodiments, the T helper peptide is conjugated to an anionic peptide or polypeptide to form an anionic peptide or polypeptide-T helper peptide conjugate. This anionic peptide or polypeptide-T helper peptide conjugate can form a complex with a cationic ionizable lipid as described elsewhere herein and can as such be encapsulated within the lipid nanoparticle.

The terms “anionic peptide” and “anionic polypeptide” as used refer to a peptide or polypeptide that has an anionic or a negative charge at physiologic pH. The anionic polypeptide may include a plurality of negatively charged amino acid residues or negatively charged unnatural amino acid residues, or a combination thereof. For example, the anionic polypeptide may include a plurality of “repeats” of negatively charged amino acid residues or negatively charged unnatural amino acid residues, or a combination thereof. The number of repeats may range from about 2 to about 50 or more. At least 20%, 30%, 40%, 50%, 60%, 70%, 80% or 90% of residues or all residues in the anionic peptide or polypeptide may be repeats of negatively charged amino acid residues or negatively charged unnatural amino acid residues, or a combination thereof. Examples of negatively charged amino acid residues are well known in the art and include, for example, aspartic acid and glutamic acid.

In embodiments, the anionic peptide or polypeptide is an oligo-L-glutamic acid, preferably deca-L-glutamic acid.

The T helper peptide may be fused to the anionic peptide or polypeptide, or may be chemically or enzymatically conjugated to the anionic peptide or polypeptide. A linker may be used between the T helper peptide and the anionic peptide or polypeptide using common techniques as known to the skilled person.

In particular embodiments, the anionic peptide or polypeptide-T helper peptide conjugate comprises or consists of the sequence set forth in SEQ ID NO: 3 (aKXVAAWTLKAAaEEEEEEEEEE), wherein a=D-alanine, X=cyclohexylalanine

As used herein, the term “adjuvant” refers to any agent that does not pose antigenic activity in and of itself, but that can be used to stimulate the immune system to increase the response to a concomitantly administered antigen or hapten. There are many suitable adjuvants, for example but not limited to, stimulators of pattern recognition receptors, such as Toll-like receptors, RIG-1 and NOD-like receptors (NLR), stimulator of interferon genes (STING), mineral salts, such as alum, alum combined with monophosphoryl lipid (MPL) A of Enterobacteria, such as Escherichia coli, Salmonella minnesota, Salmonella typhimurium, or Shigella flexneri, saponins, such as QS-21, Quil-A, ISCOMs, ISCOMATRIX™, emulsions such as MF59™, Montanide® ISA 51 and ISA 720, ASO2 (QS21+squalene+MPL®), AS15, liposomes and liposomal formulations such as ASO1, cyclic dinucleotide, amidobenzimidazoles, synthesized or specifically prepared microparticles and microcarriers such as bacteria-derived outer membrane vesicles (OMV) of N. gonorrhoeae, Chlamydia trachomatis and others, or chitosan particles, depot-forming agents, such as Pluronic® block co-polymers, specifically modified or prepared peptides, such as muramyl dipeptide, aminoalkyl glucosaminide 4-phosphates, such as RC529, or proteins, such as bacterial toxoids or toxin fragments.

In preferred embodiments, the adjuvant comprises one or more toll-like receptor (TLR) agonists. In particular, the adjuvant may comprise one or more agonists for TLRs 2, 3, 4, 5, 7, 8, 9 or any combination thereof, preferably a TLR 7 and 8 (TLR7/8) agonist. Non-limiting examples of TLR agonists include, without limitation, imidazoquinolines, adenine derivatives and immunostimulatory DNA or RNA. In particular embodiments, the adjuvant comprises an imidazoquinoline (IMDQ) compound such as, e.g., imidazoquinoline (IMDQ), imiquimod, resiquimod, imidazoquinoline amines, imidazopyridine amines, 6,7-fused cycloalkylimidazopyridine amines, and 1,2-bridged imidazoquinoline amines.

In embodiments, the adjuvant is conjugated to a lipid, and said lipid-adjuvant conjugate is encapsulated within the lipid nanoparticle through hydrophobic interaction (i.e. when aggregating the nonpolar or hydrophobic lipid structure of the conjugate). The lipid may be conjugated to the adjuvant via a covalent conjugation, for example through a linker such as a disulfide bridge, or via non-covalent interactions such as hydrogen bonds. In a preferred embodiment, the lipid-adjuvant conjugate is a lipid-SS-IMDQ conjugate, such as the conjugate of example 2.

Any lipid can be used for conjugation to the adjuvant such as, without limitation, a fatty acid, a monoglyceride, a diglyceride, a triglyceride, a steroid, a phosphatide, a phospholipid, or analogue thereof (e.g. phosphatidylcholine, lecithin, phosphatidylethanolamine, cephalin, or phosphatidylserine or analogue or portion thereof, such as a partially hydrolyzed portion thereof). The fatty acid may be a short-chain, medium-chain, or long-chain fatty acid. For example, the lipid, e.g. fatty acid, may have a C2-C60 chain, a C2-C40 chain or a C4-C40 chain, a C2-28 chain, or a C2-C12 chain or a C4-C12 chain. The fatty acid may be a saturated fatty acid or an unsaturated fatty acid, such as a monounsaturated fatty acid or a polyunsaturated fatty acid, such as an ω-3 (omega-3) or ω-6 (omega-6) fatty acid. The adjuvant may also be conjugated to two fatty acids, which may be the same or different fatty acids.

In particular embodiments, the lipid-adjuvant conjugate is 1-(4-((4-amino-2-butyl-1H-imidazo[4,5-c]quinolin-1-yl)methyl)phenyl)-3,12-dioxo-4,11-dioxa-7,8-dithia-2,13-diazahexadecane-15,16-diyl dipalmitate (lipid-S—S-IMDQ).

In embodiments, the fentanyl hapten is conjugated to the outer surface of the lipid nanoparticle via a hydrophylic, non-immunogenic polymer moiety conjugated to, e.g. covalently bound to, a lipid.

Non-limiting examples of suitable hydrophilic, non-immunogenic polymers include, for example, homopolymers selected from the group consisting of poly(alkyl-oxazoline), polysaccharide, poly(N-vinylpyrrolidone), poly(sarcosine), polyvinyl alcohol (PVA), poly(acrylic acid), poly(methacrylic acid), poly(acrylamide) and polyethylene glycol (PEG). Alternatively, or in addition, the hydrophilic, non-immunogenic polymer can be selected from heteropolymers comprising at least one monomeric subunit from two or more of the polymers selected from the group consisting of poly(alkyl-oxazoline), polysaccharide, poly(N-vinylpyrrolidone), poly(sarcosine), polyvinyl alcohol (PVA), poly(acrylic acid), poly(methacrylic acid), poly(acrylamide) and polyethylene glycol (PEG).

In preferred embodiments, the fentanyl hapten is conjugated to the outer surface of the lipid nanoparticle via a polyethylene glycol (PEG)-modified lipid.

Non-limiting examples of suitable PEGylated lipids for conjugation to the fentanyl hapten include, without limitation, PEGylated dialkylamines, PEGylated phosphatidylethanolamines, PEGylated phosphatidic acids, PEGylated ceramides, PEGylated diacylglycerols, PEGylated dialkylglycerols, and mixtures thereof. In particular embodiments, the fentanyl hapten is conjugated to a PEGylated dialkylamine.

As used herein, the term “ionizable lipid” refers to lipids having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or a neutral lipid refers to the nature of the predominant species and does not require that all of the lipid be present in the charged or neutral form. Ionizable lipids are also referred to as cationic ionizable lipids or cationic lipids herein. Advantageously, the positively charged lipids are able to associate with negatively charged compounds. Also advantageously, a positive charge on the LNP may promote association with the negatively charged cell membrane to enhance cellular uptake.

The ionizable lipid for use in the lipid nanoparticles of the invention can be any type of ionizable lipid. Exemplary ionizable lipids are disclosed in e.g. WO 2022/136641 A1 and WO 2022/207938 A1. For example, suitable ionizable lipids are ionizable amino lipids which comprise an ionizable amine headgroup, and a disulfide bond in the linker region between the head group and the one or more lipid tails.

In particular embodiments, the ionizable lipid is represented by the formula (V)

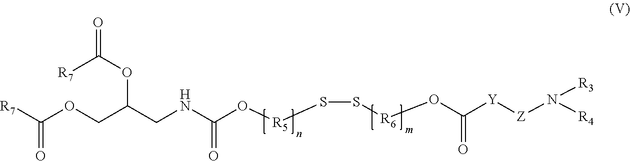

wherein:
R₃ and R₄ are each independently a —C₁₋₆ alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆ alkyl; and
each R₅ and R₆ is independently —CH₂—;
each R₇ is independently selected from —C₁₋₂₀ alkyl, —C₂₋₂₀ alkenyl, —C₂₋₂₀ alkynyl; wherein each of said —C₁₋₂₀ alkyl, —C₂₋₂₀ alkenyl, —C₂₋₂₀ alkynyl may optionally further comprise one or more heteroatoms and/or may optionally be substituted with from 1 to 3 —O—(C═O)—R₇, —(C═O)—O— R₇, —C₁₋₂₀ alkyl, —C₂₋₂₀ alkenyl, —C₂₋₂₀ alkynyl; and the total number of C atoms in both R₇ moieties together is at least 5;
m and n are each independently an integer selected from 1, 2, 3 and 4;
Y is selected from —NH— and —O—;
Z is —C₁₋₆ alkylene-.

The term “alkyl” as used herein by itself or as part of another substituent refers to a fully saturated hydrocarbon of Formula CₓH₂ₓ₊₁ wherein x is a number greater than or equal to 1. Generally, alkyl groups of this invention comprise from 1 to 20 carbon atoms. Alkyl groups may be linear or branched and may be substituted as indicated herein. When a subscript is used herein following a carbon atom, the subscript refers to the number of carbon atoms that the named group may contain. Thus, for example, C₁₋₄alkyl means an alkyl of one to four carbon atoms. Examples of alkyl groups are methyl, ethyl, n-propyl, i-propyl, butyl, and its isomers (e.g. n-butyl, i-butyl and t-butyl); pentyl and its isomers, hexyl and its isomers, heptyl and its isomers, octyl and its isomers, nonyl and its isomers; decyl and its isomers, undecyl and its isomers, dodecyl and its isomers, tridecyl and its isomers, tetradecyl and its isomers, pentadecyl and its isomers, hexadecyl and its isomers, heptadecyl and its isomers, octadecyl and its isomers, nonadecyl and its isomers, eicosanyl and its isomers.

The term “optionally substituted alkyl” refers to an alkyl group optionally substituted with one or more substituents (for example 1 to 4 substituents, for example 1, 2, 3, or 4 substituents) at any available point of attachment. Non-limiting examples of such substituents include esters, carboxylic acids, alkyl moieties, alkene moieties, alkyne moieties, . . . and the like.

The alkyl, alkene and alkyne moieties as defined herein may also further comprise one or more heteroatoms, in that for example a C atom in an alkyl, alkene or alkyne chain is replaced by a heteroatom, such as selected from N, S or O.

The term “alkenyl” or “alkene”, as used herein, unless otherwise indicated, means straight-chain, cyclic, or branched-chain hydrocarbon radicals containing at least one carbon-carbon double bond. Examples of alkenyl radicals include ethenyl, propenyl, isopropenyl, butenyl, isobutenyl, pentenyl, hexenyl, hexadienyl, be it in the terminal or internal positions and the like. Alkenyl or alkene moieties as used herein may comprise from 2 to 20 C atoms. An optionally substituted alkenyl refers to an alkenyl having optionally one or more substituents (for example 1, 2, 3 or 4), selected from those defined above for substituted alkyl.

The term “alkynyl” or “alkyne”, as used herein, unless otherwise indicated, means straight-chain or branched-chain hydrocarbon radicals containing at least one carbon-carbon triple bond. Examples of alkynyl radicals include ethynyl, E- and Z-propynyl, isopropynyl, E- and Z-butynyl, E- and Z-isobutynyl, E- and Z-pentynyl, E, Z-hexynyl, and the like. Generally alkenyl or alkyne moieties as used herein comprise from 2 to 20 C atoms. An optionally substituted alkynyl refers to an alkynyl having optionally one or more substituents (for example 1, 2, 3 or 4), selected from those defined above for substituted alkyl. T The term “cycloalkyl” by itself or as part of another substituent is a cyclic alkyl group, that is to say, a monovalent, saturated, or unsaturated hydrocarbyl group having 1, 2, or 3 cyclic structure. Cycloalkyl includes all saturated or partially saturated (containing 1 or 2 double bonds) hydrocarbon groups containing 1 to 3 rings, including monocyclic, bicyclic, or polycyclic alkyl groups. Cycloalkyl groups may comprise 3 or more carbon atoms in the ring and generally, according to this invention comprise from 3 to 15 atoms. Examples of cycloalkyl groups include but are not limited to cyclopropyl, cyclobutyl, cyclopentyl, cyclohexyl, cycloheptyl, cyclooctyl, cyclononyl, adamantanyl and cyclodecyl with cyclopropyl being particularly preferred. An “optionally substituted cycloalkyl” refers to a cycloalkyl having optionally one or more substituents (for example 1 to 3 substituents, for example 1, 2, 3 or 4 substituents), selected from those defined above for substituted alkyl.

Where alkyl groups as defined are divalent, i.e., with two single bonds for attachment to two other groups, they are termed “alkylene” groups. Non-limiting examples of alkylene groups includes methylene, ethylene, methylmethylene, trimethylene, propylene, tetramethylene, ethylethylene, 1,2-dimethylethylene, pentamethylene and hexamethylene. Similarly, where alkenyl groups as defined above and alkynyl groups as defined above, respectively, are divalent radicals having single bonds for attachment to two other groups, they are termed “alkenylene” and “alkynylene” respectively.

The term “heterocycle” as used herein by itself or as part of another group refers to non-aromatic, fully saturated or partially unsaturated cyclic groups (for example, 3 to 13 member monocyclic, 7 to 17 member bicyclic, or 10 to 20 member tricyclic ring systems, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms. An optionally substituted heterocyclic refers to a heterocyclic having optionally one or more substituents (for example 1 to 4 substituents, or for example 1, 2, 3 or 4), selected from those defined above for substituted alkyl. Non-limiting examples of heterocycle comprise: piperidinyl, azepanyl, morpholinyl.

The term “aryl” (herein also referred to as aromatic heterocycle) as used herein refers to a polyunsaturated, aromatic hydrocarbyl group having a single ring (i.e. phenyl) or multiple aromatic rings fused together (e.g. naphthalene or anthracene) or linked covalently, typically containing 6 to 10 atoms; wherein at least one ring is aromatic. The aromatic ring may optionally include one to three additional rings (either cycloalkyl, heterocyclyl, or heteroaryl) fused thereto. Aryl is also intended to include the partially hydrogenated derivatives of the carbocyclic systems enumerated herein. Non-limiting examples of aryl comprise phenyl, . . . . The aryl ring or heterocycle as defined herein can optionally be substituted by one or more substituents (for example 1 to 5 substituents, for example 1, 2, 3 or 4) at any available point of attachment. Non-limiting examples of such substituents are selected from halogen, hydroxyl, oxo, nitro, amino, hydrazine, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkoxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R^{a}, alkylthio, carboxyl, and the like, wherein R^{a} is alkyl or cycloalkyl. Where a carbon atom in an aryl group is replaced with a heteroatom, the resultant ring is referred to herein as a heteroaryl ring.

The term “heteroaryl” as used herein by itself or as part of another group refers but is not limited to 5 to 12 carbon-atom aromatic rings or ring systems containing 1 to 3 rings which are fused together or linked covalently, typically containing 5 to 8 atoms; at least one of which is aromatic in which one or more carbon atoms in one or more of these rings can be replaced by oxygen, nitrogen or sulfur atoms where the nitrogen and sulfur heteroatoms may optionally be oxidized and the nitrogen heteroatoms may optionally be quaternized. Such rings may be fused to an aryl, cycloalkyl, heteroaryl or heterocyclyl ring. Non-limiting examples of such heteroaryl, include: piridinyl, azepinyl, . . . .

An “optionally substituted heteroaryl” refers to a heteroaryl having optionally one or more substituents (for example 1 to 4 substituents, for example 1, 2, 3 or 4), selected from those defined above for substituted aryl.

The term “oxo” as used herein refers to the group ═O.

The term “alkoxy” or “alkyloxy” as used herein refers to a radical having the Formula —OR^{b} wherein R^{b} is alkyl. Preferably, alkoxy is C₁-C₁₀ alkoxy, C₁-C₆ alkoxy, or C₁-C₄ alkoxy. Non-limiting examples of suitable alkoxy include methoxy, ethoxy, propoxy, isopropoxy, butoxy, isobutoxy, sec-butoxy, tert-butoxy, pentyloxy and hexyloxy. Where the oxygen atom in an alkoxy group is substituted with sulfur, the resultant radical is referred to as thioalkoxy.

“Haloalkoxy” is an alkoxy group wherein one or more hydrogen atoms in the alkyl group are substituted with halogen. Non-limiting examples of suitable haloalkoxy include fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy, 2,2,2-trichloroethoxy; trichloromethoxy, 2-bromoethoxy, pentafluoroethyl, 3,3,3-trichloropropoxy, 4,4,4-trichlorobutoxy. The term “carboxy” or “carboxyl” or “hydroxycarbonyl” by itself or as part of another substituent refers to the group —CO₂H. Thus, a carboxyalkyl is an alkyl group as defined above having at least one substituent that is —CO₂H.

The term “alkoxycarbonyl” by itself or as part of another substituent refers to a carboxy group linked to an alkyl radical i.e. to form —C(═O)OR^{e}, wherein R^{e} is as defined above for alkyl.

The term “alkylcarbonyloxy” by itself or as part of another substituent refers to a —O—C(═O)R^{e} wherein R^{e} is as defined above for alkyl.

Whenever the term “substituted” is used herein, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture, and formulation into a therapeutic agent.

Where groups may be optionally substituted, such groups may be substituted with once or more, and preferably once, twice or thrice. Substituents may be selected from, for example, the group comprising halogen, hydroxyl, oxo, nitro, amido, carboxy, amino, cyano haloalkoxy, and haloalkyl.

As used herein the terms such as “alkyl, aryl, or cycloalkyl, each being optionally substituted with” or “alkyl, aryl, or cycloalkyl, optionally substituted with” refers to optionally substituted alkyl, optionally substituted aryl and optionally substituted cycloalkyl.

In a preferred embodiment, the cationic ionizable lipid is selected from the group comprising or consisting of 1-(azepan-1-yl)-4,13-dioxo-5,12-dioxa-8,9-dithia-3,14-diazaheptadecane-16,17-diyl dioleate (S—Ac-7-Dog) and [(4-hydroxybutyl)azanediyl]di(hexane-6,1-diyl) bis(2-hexyldecanoate) (ALC-0315).

In addition to the ionizable lipids, in particular cationic ionizable lipids, the lipid nanoparticle may further comprise a structural helper lipid.

Suitable helper lipids are generally known in the art. A preferred helper lipid is a steroid or a sterol, more preferably cholesterol. Incorporation of a steroid or a sterol in the LNP may help aggregation of other lipids in the lipid nanoparticle. As defined herein, “sterols” are a subgroup of steroids consisting of steroid alcohols. A particularly preferred sterol is cholesterol or an analogue thereof. Other examples include ergosterol and phytosterols. Other possible helper lipids are phospholipids including, without limitation, dioleoylphosphatidylethanolamine (DOPE) and 1,2-distearoyl-sn-glycero-3-phosphocholine (DSPC). In particular embodiments, the lipid nanoparticle further comprises a sterol, preferably cholesterol, or a phospholipid, preferably 1,2-distearoyl-sn-glycero-3-phosphocholine (DSPC), or any combination thereof.

In particular embodiments, the invention provides a lipid nanoparticle comprising:
the fentanyl hapten conjugated to a PEG-modified lipid (lipid-PEG-fentanyl hapten conjugate);
a PADRE conjugated to an anionic peptide or polypeptide (anionic peptide or polypeptide-PADRE conjugate);
a cationic ionizable lipid;
a lipid-conjugated TLR agonist (lipid-TLR agonist conjugate); and
a structural helper lipid selected from the group consisting of: sterols and phospholipids, preferably a sterol and a phospholipid.

In other particular embodiments, the invention provides a lipid nanoparticle comprising:
the fentanyl hapten conjugated to a PEG-modified lipid (lipid-PEG-fentanyl hapten conjugate);
a PADRE conjugated to an anionic peptide of polypeptide (anionic peptide or polypeptide conjugate);
a cationic ionizable lipid; and
a structural helper lipid selected from the group consisting of sterols and phospholipids, preferably a sterol and phospholipid.

In other particular embodiments, the invention provides a lipid nanoparticle comprising:
the fentanyl hapten conjugated to a PEG-modified lipid (lipid-PEG-fentanyl hapten conjugate);
a lipid-conjugated TLR agonist (lipid-TLR agonist conjugate);
a cationic ionizable lipid; and
a structural helper lipid selected from the group consisting of sterols and phospholipids, preferably a sterol and phospholipid.

In embodiments, the lipid nanoparticle comprises:
about 0.1 mol % to about 10.0 mol %, preferably about 0.5 mol % to about 5.0 mol %, more preferably about 1.0 mol % to about 3.5 mol % or about 1.1 mol % to about 3.0 mol %, such as about 1.5 mol %, lipid-PEG-fentanyl hapten conjugate; and/or, preferably and,
about 20.0 mol % to about 80.0 mol %, preferably about 30.0 mol % to about 70.0 mol %, more preferably about 40.0 mol % to about 65.0 mol % or about 45.0 mol % to about 60.0 mol %, such as about 50.0 mol %, cationic ionizable lipid; and/or, preferably and,
about 0.1 mol % to about 10.0 mol %, preferably about 0.5 mol % to about 5.0 mol %, more preferably about 0.9 mol % to about 3.5 mol % or about 1.1 mol % to about 3.0 mol %, such as about 1.3 mol %, lipid-TLR agonist conjugate; and/or, preferably and,
about 10.0 mol % to about 90.0 mol %, preferably about 20.0 mol % to about 70.0 mol %, more preferably about 30.0 mol % to about 65.0 mol % or about 40.0 mol % to about 55.0 mol %, such as about 47.2 mol %, structural helper lipid.

As used herein, the term “mol %” refers to a molar percentage of a lipid component based on the total moles of lipids in the LNP.

In further embodiments, the lipid nanoparticle comprises:
about 30.0 mol % to about 45.0 mol %, preferably about 35.0 mol % to about 45.0 mol %, about 36.0 mol % to about 42.0 mol % or about 37.0 mol % to about 40.0 mol %, such as about 38.5 mol %, sterol; and/or, preferably and,
about 5.0 mol % to about 15.0 mol %, preferably about 6.0 mol % to about 12.0 mol %, about 7.0 mol % to about 11.0 mol % or about 7.0 mol % to about 10.0 mol %, such as about 8.7 mol %, phospholipid.

In embodiments, the LNP has an N/P ratio between 3:1 to 30:1, preferably between 4:1 and 20:1 or between 4:1 and 10:1 such as about 5:1. The “N/P ratio” is defined herein as the molar ratio of cationic ionizable nitrogen atoms in the cationic ionizable lipid to anionic amino acids in the PADRE conjugate.

As noted above, lipids make up the major part of the structural materials of the LNPs described herein. In preferred embodiments the LNP is free of polymer materials or the LNP does not comprise lipid-polymer nanoparticles.

In preferred embodiments, the LNP comprises a lipid core and a lipid layer surrounding the core.

In an embodiment the lipid core comprises one or more complexes comprising a conjugate of the T helper peptide as described herein and an anionic peptide or polypeptide, preferably an anionic peptide or polypeptide-PADRE conjugate as described herein, and a cationic ionizable lipid as described herein. The lipid layer preferably comprises a conjugate of the fentanyl hapten and a hydrophilic polymer-conjugated lipid, preferably a PEG-modified lipid. A conjugate of the adjuvant as described herein and a lipid, preferably a lipid-TLR agonist conjugate as described herein, may be present in the lipid core and/or in the lipid layer.

In embodiments, the density of the fentanyl hapten ranges from 1 to 10¹² fentanyl hapten molecules per lipid nanoparticle, preferably from about 1 to about 10⁹ fentanyl hapten molecules per nanoparticle such as from about 10 to about 10⁹, from about 10² to about 10⁹ or from about 10³ to about 10⁹ fentanyl hapten molecules per nanoparticle. In embodiments, the lipid nanoparticle comprises about 0.1 mol % to about 10.0 mol %, preferably about 0.5 mol % to about 5.0 mol %, more preferably about 1.0 mol % to about 3.5 mol % or about 1.1 mol % to about 3.0 mol %, such as about 1.5 mol %, of a conjugate of the fentanyl hapten and a polymer-conjugated lipid as taught herein, in particular a lipid-PEG-fentanyl hapten conjugate.

In embodiments, the LNP has a mean diameter of about 10 to about 999 nm, preferably from about 10 to about 500 nm, more preferably from about 50 to about 200 nm, even more preferably from about 100 to about 150 nm. Techniques for measuring the size of nanoparticles are known to the skilled person and include, for example, dynamic light scattering. In embodiments, the mean diameter of the LNP is determined by dynamic light scattering.

A further aspect of the invention provides a pharmaceutical composition, comprising one or more LNPs as described herein and a pharmaceutically acceptable carrier. Such pharmaceutical compositions are particularly suitable as vaccines. In particular embodiments, the pharmaceutical composition is a vaccine composition or a vaccine.

The term “pharmaceutically-acceptable carrier” means one or more compatible solid or liquid filler, diluents or encapsulating substances which are suitable for administration to a subject contemplated by the present application. The term “carrier” denotes an organic or inorganic ingredient, natural or synthetic, with which the lipid nanoparticles and any other optional agent(s) are combined to facilitate administration. The pharmaceutical composition may further comprise one or more pharmaceutically acceptable ingredients, as known to the skilled person, including, but not limited to polymeric excipients, for instance chitosan derivatives or salts thereof; lipid excipients, such as phospholipids; surfactants; solvents; buffering agents, and the like. The components of the pharmaceutical compositions are commingled in a manner that precludes interaction that would substantially impair their desired pharmaceutical efficiency.

The type of carrier and other ingredients will vary depending on the mode of administration. When it is desirable to deliver the pharmaceutical compositions as envisaged herein systemically, it may be formulated for parenteral administration by injection, e.g., by bolus injection or continuous infusion. Formulations for injection may be presented in unit dosage form. Pharmaceutical parenteral formulations may include aqueous solutions of the ingredients. Aqueous injection suspensions may contain substances which increase the viscosity of the suspension, such as sodium carboxymethyl cellulose, sorbitol, or dextran. Alternatively, injection suspensions may be prepared as oil-based suspensions such as are known in the art or that will be readily apparent to those of ordinary skill in the art based on this disclosure.

In the context of the present invention, the term “vaccine” refers to any formulation intended to provide adaptive immunity (antibody and/or T cell response) against fentanyl, e.g. to prevent or treat a fentanyl abuse disorder, a fentanyl addiction or a fentanyl overdose. To this end, a vaccine as described herein contains a fentanyl hapten conjugated to a LNP as described herein, against which an adaptive immune response is raised. Vaccines can be prophylactic (e.g., to prevent or ameliorate the effects of future fentanyl abuse or overdose) or therapeutic (e.g, to actively treat or reduce the symptoms of an ongoing disease or disorder associated with fentanyl abuse).

The vaccines of the present invention may be useful for the prevention or treatment of an addiction to fentanyl and/or fentanyl abuse disorder and/or a fentanyl overdose. Without wishing to be bound by any theory, the vaccines may induce a fentanyl-specific antibody response that may block the addictive fentanyl from passing the blood-brain barrier, thereby reducing or eliminating the fentanyl-induced alterations in brain chemistry, which is the source of fentanyl-addiction. In addition, the induced “immunoantagonists” may effectively minimize concentrations of fentanyl at the sites of action. As a result, overdose potential of fentanyl may be reduced and fentanyl abuse disorders may be ameliorated.

A further aspect of the invention thus provides the lipid nanoparticle or the pharmaceutical composition as described herein for use in medicine.

A further aspect of the invention provides a LNP or a pharmaceutical composition as described herein for use in a method for inducing an immune response against fentanyl in a subject, said method comprising administering the subject an immunologically effective amount of the lipid nanoparticle or the pharmaceutical composition. A related aspect is directed to a method for inducing an immune response against fentanyl in a subject, said method comprising administering to the subject an immunologically effective amount of the lipid nanoparticle or the pharmaceutical composition as described herein. Also provided is use of a LNP as or a pharmaceutical composition as described herein for the manufacture of a medicament for inducing an immune response against fentanyl in a subject.

With “immunologically effective amount” is meant herein the amount of lipid nanoparticle effective to induce an immune response in a subject against a subsequent exposure to fentanyl. Levels of induced immunity can be determined, e.g., by measuring amounts of neutralizing secretory and/or serum antibodies, e.g., by enzyme-linked immunosorbent, or microneutralization assay.

Another aspect of the invention provides a LNP or a pharmaceutical composition as described herein for use in the prevention or treatment of a fentanyl abuse disorder, a fentanyl addiction or a fentanyl overdose in a subject, said method comprising administering a therapeutically effective amount of the lipid nanoparticle or the pharmaceutical composition to the subject. A related aspect is directed to a method for the prevention or treatment of a fentanyl abuse disorder, a fentanyl addiction or a fentanyl overdose in a subject, said method comprising administering a therapeutically effective amount of the lipid nanoparticle or the pharmaceutical composition as described herein to the subject. Also provided is use of a LNP or a pharmaceutical composition as described herein for the manufacture of a medicament for the prevention or treatment of a fentanyl abuse disorder, a fentanyl addiction or a fentanyl overdose in a subject.

As used herein, the terms “subject” or “patient” are generally used interchangeably and refer to animals, preferably warm-blooded animals, more preferably vertebrates, even more preferably mammals, still more preferably primates, and specifically includes human patients and non-human mammals and primates. Preferred subjects or patients are human subjects.

The terms “treat” or “treatment” encompass both the therapeutic treatment of an already developed disease or condition, such as the therapy of an already developed fentanyl abuse disorder, a fentanyl addiction or a fentanyl overdose, as well as prophylactic or preventive measures, wherein the aim is to prevent or lessen the chances of incidence of an undesired affliction, such as to prevent occurrence, development and progression of a fentanyl abuse disorder, a fentanyl addiction or a fentanyl overdose.

A ‘therapeutic amount’ or ‘therapeutically effective amount’ as used herein refers to the amount of lipid nanoparticle effective to treat a disease or disorder in a subject, i.e., to obtain a desired local or systemic effect. The term thus refers to the quantity of lipid nanoparticle that elicits the biological or medicinal response in a tissue, system, animal, or human that is being sought by a researcher, veterinarian, medical doctor or other clinician. Such amount will typically depend on the lipid nanoparticle and the severity of the disease or disorder, but can be decided by the skilled person, possibly through routine experimentation.

Administration of the pharmaceutical composition described herein may be once, twice, three times, four times, or more times at appropriate time intervals to evoke and maintain the desired anti-fentanyl immune response. The time intervals between different doses (e.g., between the primary dose and second dose, or between the second dose and a third dose) may not be the same, and the time interval between each two doses may be determined independently. The dose of the vaccine composition, the number of doses administered to the subject, and the time intervals between two doses of the vaccine composition may be determined by a person skilled in the medical art. An appropriate dose and a suitable duration and frequency of administration will be determined by factors such as the condition of the patient, age of the patient, the type and severity of the patient's disease or disorder to be treated or prevented, the particular composition of the LNP, and the method of administration. Optimal doses may generally be determined using experimental in vitro assays, in vivo animal models, and/or human clinical trials. When administered in a liquid form, suitable dose sizes may vary with the size (i.e., weight or body mass) of the patient, but will typically range from about 100 μl to about 1 ml, such as about 100 μl, about 200 μl, about 300 μl, about 400 μl, about 500 μl, about 600 μl, about 700 μl, about 800 μl, about 900 μl, or about 1 ml (comprising an appropriate dose) for a 10-100 kg subject. The use of the minimum dosage that is sufficient to provide effective therapy and/or prophylaxis is usually preferred. Patients may generally be monitored for therapeutic or prophylactic effectiveness using assays suitable for the condition being treated or prevented, which assays will be familiar to those having ordinary skill in the art and are described herein.

Any route of administration can be used to deliver the composition to the subject. Indeed, although more than one route can be used to administer the composition, a particular route can provide a more immediate and more effective reaction than another route. For example, the vaccine compositions described herein may be administered via a route including parenteral, oral, enteral, transdermal/transmucosal, and inhalation. The term enteral, as used herein, is a route of administration in which the agent is absorbed through the gastrointestinal tract or oral mucosa, including oral, rectal, and sublingual. The term parenteral, as used herein, describes administration routes that bypass the gastrointestinal tract, and are typically administered by injection or infusion, including intraarterial, intradermal, subdermal, intramuscular, intranasal, intraocular, intraperitoneal, intravenous, subcutaneous, submucosal, intravaginal, intrasternal, intracavernous, intrathecal, intrameatal, and intraurethral injection. The term transdermal/transmucosal, as used herein, is a route of administration in which the agent is administered through or by way of the skin, including topical. The term inhalation encompasses techniques of administration in which an agent is introduced into the pulmonary tree, including intrapulmonary or transpulmonary and includes intranasal administration. Preferably, the composition is administered via injection.

The lipid nanoparticles of the present invention may be prepared in accordance with the protocols as specified in the Examples section. More generally, the LNP's may be prepared by combining the lipid components in suitable concentrations as described elsewhere herein in an organic solvent, preferably an alcoholic vehicle such as ethanol. Thereto, an aqueous composition comprising the T helper peptide conjugated to an anionic peptide or polypeptide is added, and subsequently mixed. Upon removal of the organic solvent from the mixture, e.g. by dialysis or other suitable techniques known to the skilled person, the LNPs are obtained.

Yet another aspect of the invention provides a method for preparing a lipid nanoparticle comprising:
mixing an aqueous composition comprising the T helper peptide conjugated to an anionic peptide or polypeptide with a lipid composition comprising a cationic ionizable lipid, the fentanyl hapten conjugated to a lipid via a hydrophilic, non-immunogenic polymer, preferably the fentanyl hapten conjugated to a PEG-modified lipid, a structural helper lipid, and/or the adjuvant conjugated to a lipid in a suitable organic solvent, such as ethanol, to form a mixture; and
removing the organic solvent from the mixture, thereby obtaining the lipid nanoparticle.

In embodiments, the method comprises the step:
preparing an aqueous composition comprising the T helper peptide conjugated to an anionic peptide or polypeptide;
preparing a lipid composition comprising the cationic ionizable lipid, the fentanyl hapten conjugated to a lipid via a hydrophilic, non-immunogenic polymer, preferably the fentanyl hapten conjugated to a PEG-modified lipid, the structural helper lipid, and/or the adjuvant conjugated to a lipid in a suitable organic solvent, such as ethanol;
mixing said aqueous composition with said lipid composition to form a mixture; and
removing the organic solvent from the mixture, thereby obtaining the lipid nanoparticle.

The above aspects and embodiments are further supported by the following non-limiting examples.

### EXAMPLES

NMR results shown herein were produced by a Bruker 300 or 400 MHz FT NMR spectrometer. Low resolution mass spectrometry (LRMS) results were obtained using an electrospray ionization mass spectrometers (ESI-MS). Both NMR and MS data were analyzed by MestRenova software.

### Example 1: Synthesis of Lipid-PEG-Fentanyl

Lipid-PEG-fentanyl was chemically synthesized according to the following scheme:

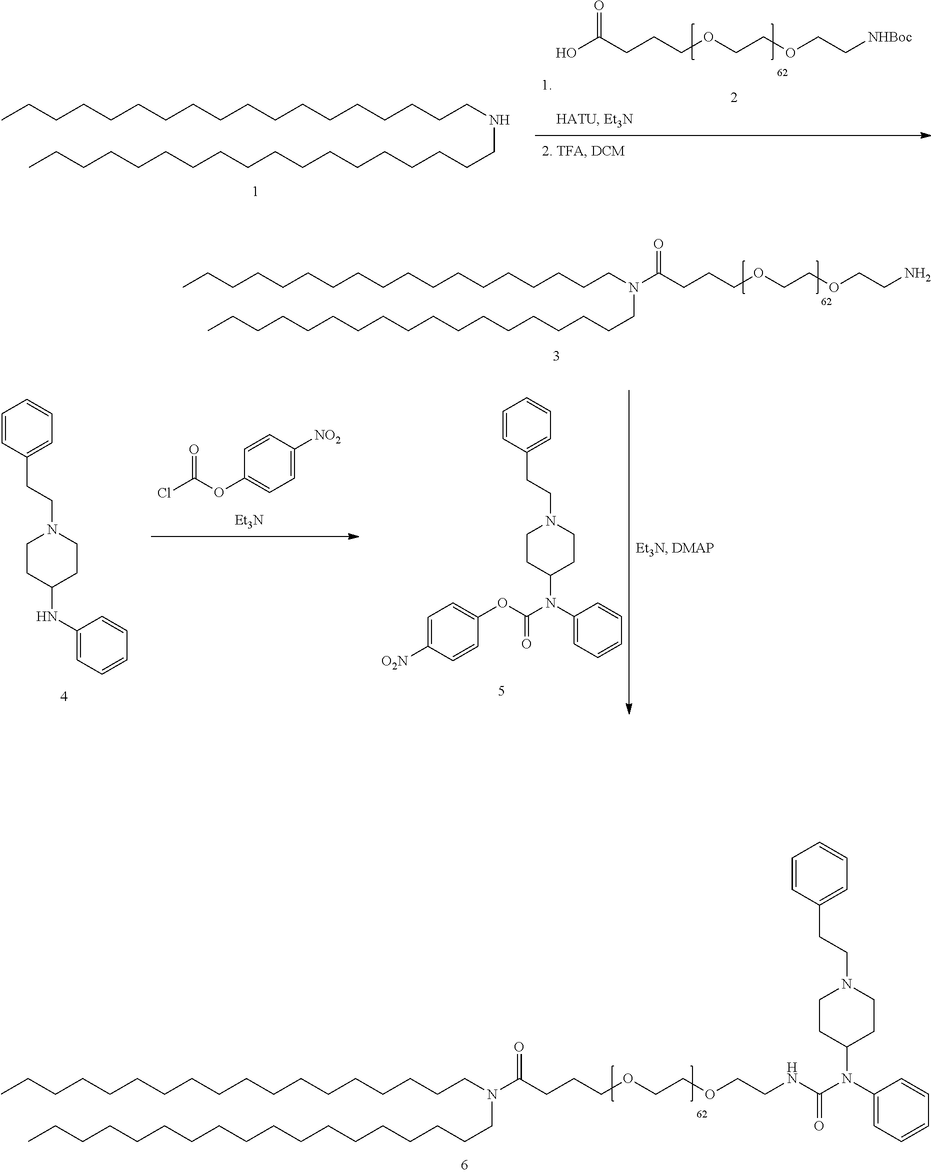

### Synthesis of Lipid-PEG-Fentanyl

### 1.1 Synthesis of Compound 3

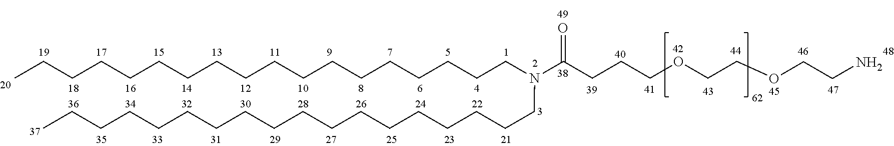

Compound 3 from the Synthesis of Lipid-PEG-Fentanyl

In a round bottom flask, butyloxycarbonyl (Boc)-protected-amino-PEG-acid (0.50 g, 0.166 mmol, 1.0 equiv.) was dissolved in 5 mL of anhydrous dimethylformamide (DMF). Next, 1-[Bis(dimethylamino)methylene]-1H-1,2,3-triazolo[4,5-b]pyridinium 3-oxide hexafluorophosphate (HATU) (0.069 g, 0.18 mmol, 1.1 equiv.) and triethylamine (Et₃N) (35 μL, 1.5 equiv.) were added to the stirring mixture of the Boc-amino-PEG-acid in DMF. After stirring for 5 minutes at room temperature, a solution of dioctadecyl amine (104.5 mg, 0.22 mmol, 1.2 equiv.) in 5 mL of anhydrous chloroform (CHCl₃) was added. After stirring overnight at room temperature, the resulting mixture was concentrated. Without further purification, the mixture was dissolved in dichloromethane (DCM) (5.0 mL) followed by adding trifluoroacetic acid (2.0 mL). After stirring at room temperature for 2 hours, the mixture was concentrated and the resulting crude product (dissolved in ethanol) was then transferred into a dialysis membrane (Molecular weight cut-off (MWCO): 1 kiloDalton (KD)) and dialyzed against water supplemented with 0.015M ammonia for three days (1 mL ammonia in 1 L distilled H₂O). After lyophilization, the product was obtained as a colourless powder (yield was 91%).

Nuclear magnetic resonance (NMR): ¹H NMR (400 MHz, Chloroform-d) δ ppm 3.77-3.70 (m, 2H, H46), 3.69-3.45 (m, 208H, H43 and H44), 3.44-3.34 (m, 4H, H41 and H47), 3.23-3.10 (m, 4H, H1 and H3), 2.31 (m, 2H, H39), 1.86 (m, 2H, H40), 1.50-1.39 (m, 4H, H4 and H21), 1.28-1.10 (m, 60H, H5-H19 and H22-H36), 0.88 (m, 6H, H20 and H37).

### 1.2. Synthesis of Compound 5

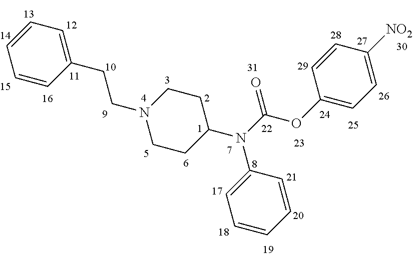 [SMILES: O=C(Oc1ccc([N+](=O)[O-])cc1)N(c1ccccc1)C1CCN(CCc2ccccc2)CC1]

Compound 5 from the Synthesis of Lipid-PEG-Fentanyl

N-[1-(2-phenylethyl)-4-piperidinyl]aniline (4) (1 equiv.) was dissolved in DCM in a round-bottom flask equipped with a stirring bar. The solution was treated sequentially with 4-nitrophenyl chloroformate (1.5 equiv.) and Et₃N (2 equiv.) at ambient temperature overnight. The resulting oily mixture was purified by flash column chromatography (1:1→7:3 ethyl acetate (EtOAc)/hexane) to give compound 5 as a light-yellow oil (yield was 82%).

NMR: ¹H NMR (400 MHz, Chloroform-d) δ ppm 8.19 (d, J=8.7 Hz, 2H, H26 and H28), 7.44-7.35 (m, 2H, H17 and H21), 7.31-7.24 (2H, H25 and H29), 7.22-7.15 (m, 8H, H12-16 and H18-20), 4.31 (s, 1H, H1), 3.15-2.97 (brd, 2H, H9), 2.88-2.67 (2H, H10), 2.67-2.46 (2H, H3 or H5), 2.3-2.06 (2H, H3 or H5), 2.04-1.80 (2H, H2 or H6), 1.79-1.56 (2H, H2 or H6). Low resolution mass spectrometry (LRMS) (ESI-MS) (m/z): calculated for [M+NH₄]⁺ (C₂₆H₂₈O₄N₃⁺) requires 446.2, found: 446.2.

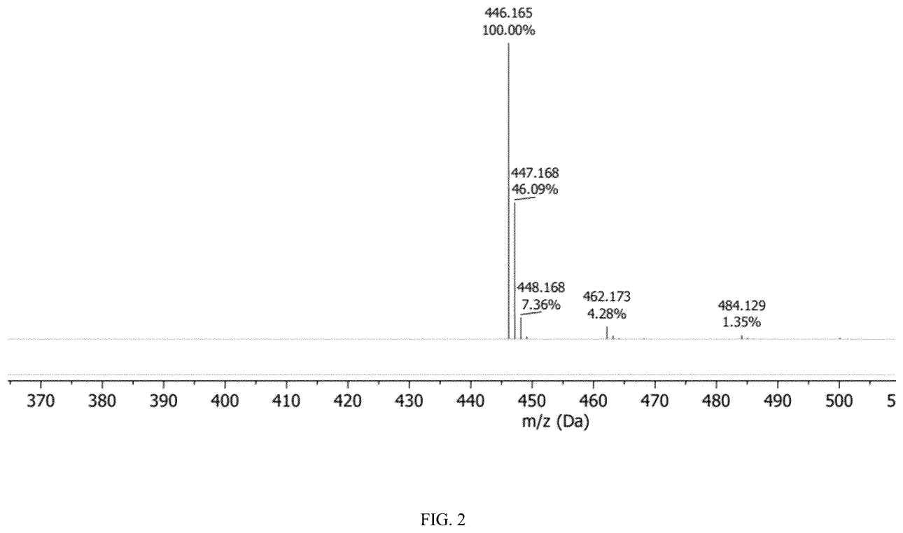

FIG. 2 shows the mass spectrometry (MS) analysis of compound 5.

### 1.3. Synthesis of Compound 6: Lipid-PEG-Fentanyl Hapten

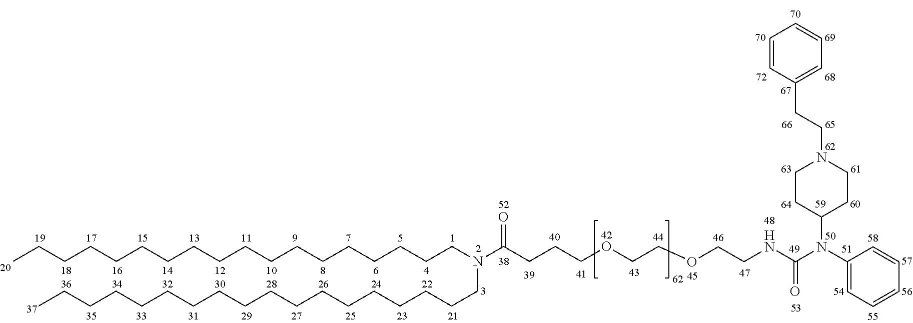

### Lipid-PEG-Fentanyl Hapten

NH₂-PEG-diC₁₈ (1.0 equiv.) was dissolved in DMF in a round-bottom flask equipped with a stirring bar. The solution was treated sequentially with Et₃N (2.0 equiv.) and 4-dimethylaminopyridine (DMAP) (0.1 equiv.) at ambient temperature. The resulting suspension was vigorously stirred at 80° C. overnight. The resulting crude mixture was then transferred into a dialysis membrane (MWCO: 1 KD) and dialyzed against water supplemented with 0.1% v/v ammonia for three days. The final product was obtained as light-yellow powder after lyophilization (yield was 87.5%).

NMR: ¹H NMR (400 MHz, Chloroform-d) δ ppm 7.90-7.81 (1H, H48), 7.54-7.40 (4H), 7.32-7.21 (6H overlapped with CHCl₃ peaks), 4.41-4.39 (1H, H59), 3.64 (brd, 195H, H41, H43 and H44), 3.11-3.0 (10H, H1, H3, H47, H61a and H63a), 2.84-2.79 (2H, H65 or H66), 2.44-2.32 (4H, H39 and H65 or H66), 2.19-2.08 (2H, H61 b and H63b), 1.98-1.88 (4H, H40, H60a and H64a), 1.63-1.50 (4H, H4 and H21, overlapped with water peaks), 1.40-1.20 (60H, H5-19 and H22-36), 0.92-0.80 (6H, H20 and H37).

### Example 2: Synthesis of Lipid-SS-IMDQ

Lipid-SS-IMDQ was synthesized according to the following scheme:

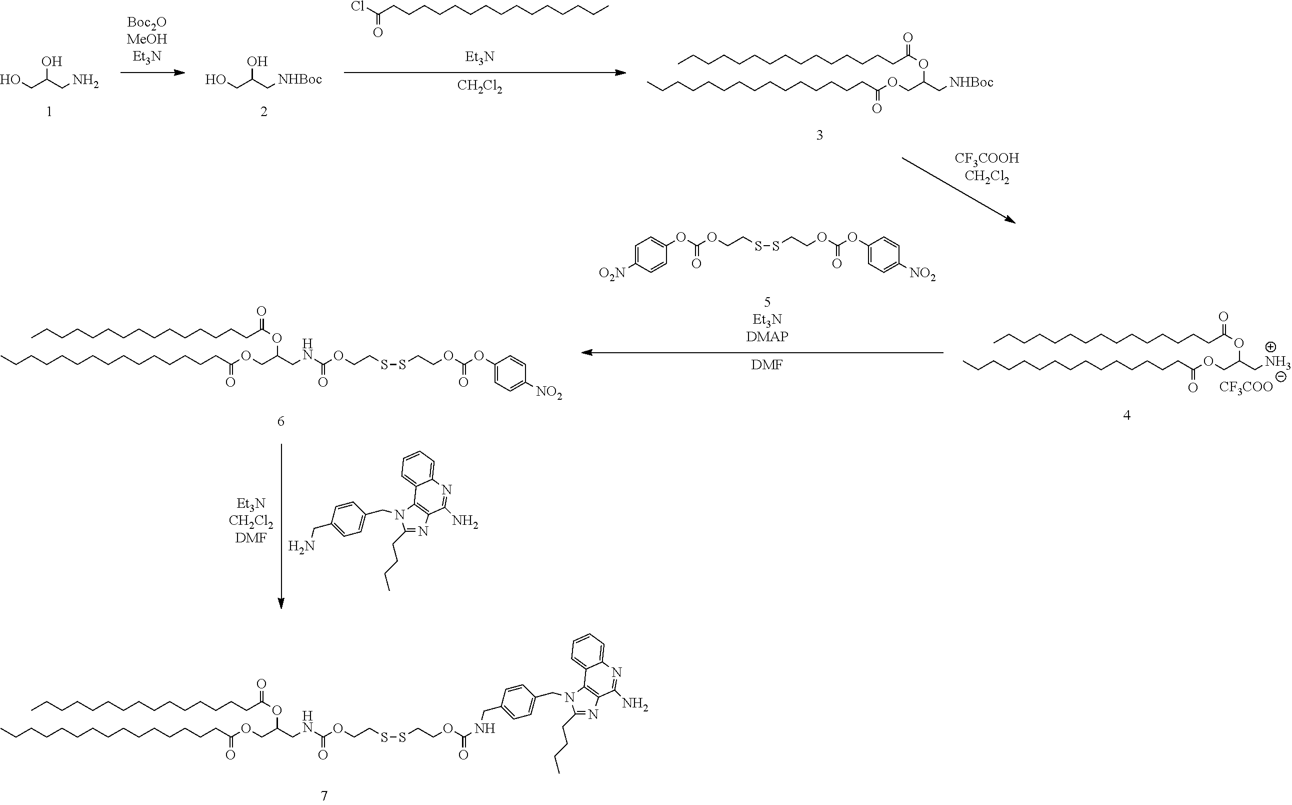

Synthesis of lipid-SS-IM DO

### 2.1 Synthesis of Compound 2

To a stirred solution of 3-aminopropane-1,2-diol 1 (5.00 g, 54.94 mmol, 1.0 equiv.) in methanol (MeOH)/Et₃N (50 mL/15.33 mL) at room temperature was dropwise added the solution of Di-tert-butyldicarbonate (Boc₂O) (14.39 g, 65.93 mmol, 1.2 equiv.) in MeOH (15 mL). The mixture was then stirred at room temperature for another 3 h. Afterwards, the solvent was removed, and the residue was redissolved in ethyl acetate (EtOAc), and washed by less water 2 times (avoiding washing too much, otherwise the product will be washed away) and less brine 1 time and dried over sodium sulphate (Na₂SO₄) overnight, filtered and concentrated.

¹H NMR showed the product 2 (bright oil) was clean enough (purity>95%) and used without further purification in the next step. (The yield was 81.43%).

### 2.2 Synthesis of Compound 3

To a stirred solution of 3-(Boc-amino)-1,2-propanediol 2 (3.00 g, 15.69 mmol, 1.0 equiv.) and palmitoyl chloride (10.47 mL, 34.52 mmol, 2.2 equiv.) in dichloromethane (CH₂Cl₂) (60 mL) at 0° C. was dropwise added Et₃N (5.47 mL, 39.23 mmol, 2.5 equiv.). The reaction mixture was vigorously stirred at room temperature overnight. Then the reaction was quenched by saturated sodium carbonate (Na₂CO₃) (aq.). The organic phase was separated, washed by brine, dried over Na₂SO₄, filtered and concentrated. The resulting residue was purified by silica gel column chromatography (hexane/ethyl acetate=9:1) to afford compound 3 as a white solid (1.00 g, the yield was 9.54%).

### 2.3 Synthesis of Compound 4

Compound 3 was then dissolved in the mixed solvent of CH₂Cl₂/trifluoroacetic acid (CF₃COOH) (8 mL/18 mL). After stirring at room temperature for 1 h, the solvent was removed under reduced pressure, and the crude product 4 was further dried under vacuum and used without further purification (0.91 g, yield was 100%).

### 2.4 Synthesis of Compound 5

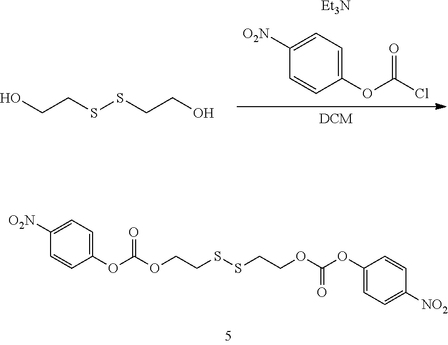

Synthesis of Compound 5 from the Synthesis of Lipid-SS-IMDQ

2,2′-disulfanediylbis(ethan-1-ol) (3.19 g, 20.68 mmol, 1.0 equiv.) and Et₃N (6.92 mL, 49.63 mmol, 2.4 equiv.) were dissolved in hydrous DCM (50 mL). Then at room temperature, the solution of 4-nitrophenyl carbonochloridate (10.00 g, 49.63 mmol, 2.4 equiv.) in DCM 40 mL was dropwise added. The mixture was then stirred for another 4 h. Then the organic phase was washed 3 times by saturated Na₂CO₃ (aq.), 1 time by brine, dried over Na₂SO₄, filtered and concentrated. The product was purified by silica gel column chromatography (hexane/ethyl acetate=15:1 to 10:1) to give the product 5 as a colorless solid (50% yield).

### 2.5 Synthesis of Compound 6

Compound 4 (0.65 g, 1.15 mmol, 1.0 equiv.), compound 5 (1.11 g, 2.30 mmol, 2.0 equiv.), DMAP (0.04 g, 0.34 mmol, 0.3 equiv.) and Et₃N (0.32 mL, 2.30 mmol, 2.0 equiv.) were dissolved in DMF (39 mL) at room temperature and the resulting mixture was stirred overnight. Solvent was removed under reduced pressure, and the mixture was purified by silica gel column chromatography (hexane/ethyl acetate=5:1) to give the product 6 as a light-yellow oil (71.3% yield).

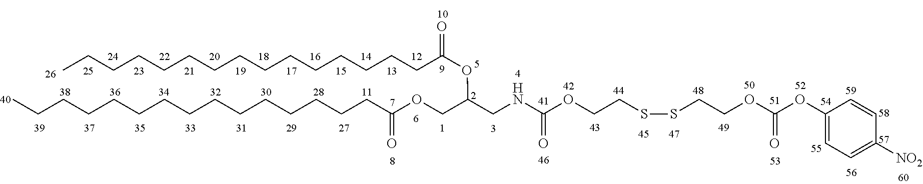 [SMILES: CCCCCCCCCCCCCCCC(=O)OCC(CNC(=O)OCCSSCCOC(=O)Oc1ccc([N+](=O)[O-])cc1)OC(=O)CCCCCCCCCCCCCCC]

Compound 6 from the Synthesis of Lipid-SS-IMDQ

¹H NMR (400 MHz, Chloroform-d) δ 8.29 (dd, 2H, H56 and H58), 7.39 (dd, 2H, H55 and H59), 5.08 (m, 1H, H2), 4.26 (m, 2H, H1), 4.12 (dd, 4H, H43 and H49), 3.40 (m, 2H, H3), 3.00 (m, 4H, H44 and H48), 2.31 (t, 4H, H11 and H12), 1.60 (dt, 4H, H13 and H27), 1.25 (m, 48H, H14-H25 and H28-H39), 0.87 (t, 6H, H26 and H40) ppm. LRMS (ESI-MS) (m/z): calculated for [M+NH₄]⁺ (C₄₇H₈₄O₁₁N₃S₂⁺) requires 930.6, found: 930.5.

### 2.6 Synthesis of Compound 7 (Lipid-SS-IMDQ)

IMDQ (5.00 mg, 13.46 μmol, 1.0 equiv.), compound 6 (36.85 mg, 40.37 μmol, 3.0 equiv.), Et₃N (0.05 mL), DCM (0.8 mL) and DMF (0.5 mL) were mixed up at room temperature and the resulting mixture was stirred for 0.5 h. Solvent was removed under reduced pressure, and the mixture was purified by preparative thin-layer chromatography (TLC) (DCM/MeOH/30% Ammonium Hydroxide=90:10:1) to give the product 7 as a colorless film (45.5% yield).

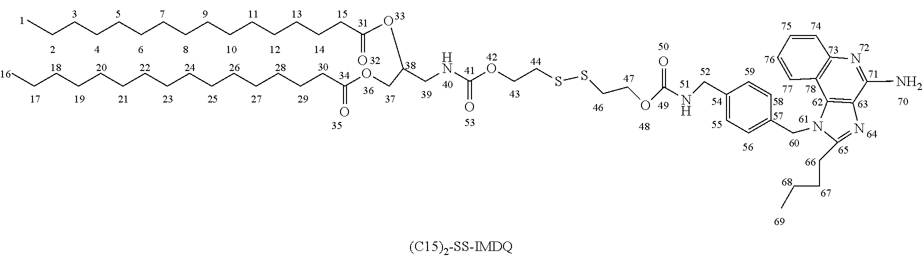 [SMILES: CCCCCCCCCCCCCCCC(=O)OCC(CNC(=O)OCCSSCCOC(=O)NCc1ccc(Cn2c(CCCC)nc3c(N)nc4ccccc4c32)cc1)OC(=O)CCCCCCCCCCCCCCC]

### Compound 7 or Lipid-S—S-IMDQ.

Yield: 45.5%. Colorless film. ¹H NMR (400 MHz, Chloroform-d) δ 7.84 (dd, 1H, H77), 7.70 (dd, 5H, H74), 7.46 (t, 1H, H75), 7.27 (dd, 2H, H55 and H59), 7.17 (t, 1H, H76), 7.01 (dd, 2H, H56 and H58), 6.44 (s, 2H, H70), 5.72 (s, 2H, H60), 5.36 (t, 1H, H51), 5.17 (t, 1H, H40), 5.08 (m, 1H, H38), 4.34-4.28 (m, 6H, H43, H47 and H52), 4.23-4.08 (m, 2H, H37), 3.37 (m, 2H, H39), 2.88 (m, 6H, H44, H46 and H66), 2.29 (t, 4H, H15 and H30), 1.80 (dt, 2H, H67), 1.59 (dt, 4H, H14 and H29), 1.44 (m, 2H, H68), 1.25 (m, 48H, H2-H13 and H17-H28), 0.94 (t, 3H, H69), 0.88 (t, 6H, H1 and H16) ppm. LRMS (ESI) (m/z): calculated for [M+H]⁺ (C₆₃H₁₀₁O₈N₆S₂⁺) requires 1133.7, found: 1133.4.

### Example 3: Design of PADRE-E10 and Determination of Encapsulation Efficiency

### 3.1. PADRE-E10 Design

The pan HLA DR-binding epitope PADRE was extended by a 10-decaglutamate amino acid sequence to provide the peptide with an overall anionic charge to allow for electrostatic complexation with an ionizable lipid. The amino acid sequence of PADRE-E10 is shown below:
aKXVAAWTLKAAaEEEEEEEEEE, wherein a=D-alanine and X=cyclohexylalanine (SEQ ID NO:3)

### 3.2. PADRE Encapsulation Efficiency in LNP

Fentanyl-LNP with (PADRE-E10) and Fentanyl-LNP without (PADRE-E10), as a control, were subjected to high-speed centrifugation (Eppendorf centrifuge 5424R, 20,000 g, 30 min, 4° C.). Then, supernatant was aspirated and transferred to a 96-well plate. The unencapsulated PADRE peptide was quantified by the BCA Protein Assay (Thermo Scientific) according to the manufacturer's instructions. Briefly, BCA working reagent was added and the plates were placed on a shaker. After 30 min incubation at 37° C., the absorbance of all samples was measured at 562 nm on a Perkin Elmer Ensight multimode plate reader. Fentanyl-LNP without (PADRE-E10) served as a blank control. The concentration of non-encapsulated PADRE-E10 in the supernatant was calculated using a calibration curve of unformulated PADRE-E10. The encapsulation efficiency of PADRE-E10 was calculated according to the following equation and the results are shown in Table 1:

$$Encapsulation efficiency = ( ( Total PADRE - E 10 - Free PADRE - E 10 ) / Total PADRE - E 10 ) \times 100 \% .$$

### Example 4: LNP Formulation of PADRE-E10, Lipid-SS-IMDQ and Lipid-PEG-Fentanyl

Fentanyl LNPs were produced by rapid mixing under vigorous stirring of an aqueous solution of PADRE-E10 as synthesized in example 3 in 5 mM acetate buffer at pH 4.0 with an ethanolic solution containing ionizable lipid S—Ac-7-Dog as described in WO 2022/136641 A1, cholesterol, DSPC, C15-SS-IMDQ as synthesized in example 2 and Lipid-PEG-Fentanyl as synthesized in example 1 at a molar ratio of 50:38.5:8.7:1.3:1.5. Fentanyl-LNP formulations were prepared at an N/P (N: mole of ionizable cationic nitrogen atoms in the ionizable lipid; P: mole of anionic amino acid in PADRE-E10) of 5:1. After mixing, LNPs were subjected to dialysis in a dialysis cassette (3.5K MWCO, Thermo Scientific, Rockford, U.S.A) against PBS to remove ethanol. LNPs were concentrated using Amicon Ultra centrifugal filters (100 kD MWCO, Merck Millipore Ltd., Belgium). The final LNP formulations were then stored at 4° C. until further use. For the in vivo application, a single dose of resulting fentanyl-LNP contained 10 μg (in 100 μL of PBS) equivalent amount of fentanyl hapten, IMDQ, and 25 μg of PADRE.

TABLE 1
Composition and physicochemical characterization of anti-fentanyl
LNP as determined by dynamic light scattering (Zetasizer
Nano-ZS, Malvern Panalytical Ltd., UK).
Anti-fentanyl LNP characterization

| | |
|---|---|
| Composition and molar ratio | S-Ac-7-Dog:cholesterol:DSPC:lipid-PEG- |
| | Fentanyl:lipid-IMDQ (50:38.5:8.7:1.5:1.3) |
| particle size (nm) | 113 ± 10 |
| zetal potential | 0.108 |
| PADRE encapsulation | 64% |
| efficiency | |

### Example 5: Immunization Studies and Antibody Titer Determination by ELISA Assay

### 5.1 Immunization of Mice

BALB/c mice, aged 7-8 weeks, were anesthetized by inhalation of isoflurane and immunized by intramuscular injection in the gastrocnemius muscle of different types of fentanyl-containing LNP formulations that contained 10 μg equivalent amount of fentanyl hapten, and/or 10 μg IMDQ, and/or 25 μg of PADRE in a final volume of 100 μL in PBS. The control mice cohort received equal volume of PBS. The primary immunization was performed at week 0 and the booster immunizations were given in mice at week 2, 4 and 19.

### 5.2 Fentanyl-Specific Antibody Titer Determination by ELISA Assay

Fentanyl-specific antibody titers were determined by enzyme-linked immunosorbent assay (ELISA) assay with a commercially available ELISA buffer kit (Invitrogen) according to the manufacturer's instructions. Briefly, uncoated 96-well ELISA plates (Biolegend) were coated by addition of 30 ng of fentanyl-BSA conjugate to each well, followed by overnight incubation at 4° C. and subsequently blocking with assay buffer for 1 h at room temperature. Five-fold serial dilutions of the serum samples were made (starting from a 100-fold dilution) and 100 μL of these dilutions was added per well. After 1 h of incubation at room temperature, the plates were washed four times with wash solution. Next, 100 μL of goat anti-mouse Immunoglobulin G (IgG) horseradish peroxidase (HRP)-conjugated secondary antibody (Tebu-bio), goat anti-mouse IgG1 HRP-conjugated secondary antibody (Invitrogen), or goat anti-mouse IgG2a HRP-conjugated secondary antibody (Invitrogen) in 1:3000 dilution was added to the wells and incubated for 1 h at room temperature. Subsequently, the wells were washed five times and then incubated with 100 μL of 3,3′,5,5′-Tetramethylbenzidine (TMB) substrate at room temperature. The enzymatic conversion of TMB was stopped after 15 min by adding 100 μL of stop solution containing 0.16 M sulfuric acid, and the absorbance was measured at 450 nm using an EnSight microplate reader (PerkinElmer, United States). The antibody titers were defined as the area under the curve (AUC) of the ELISA dilution curves.

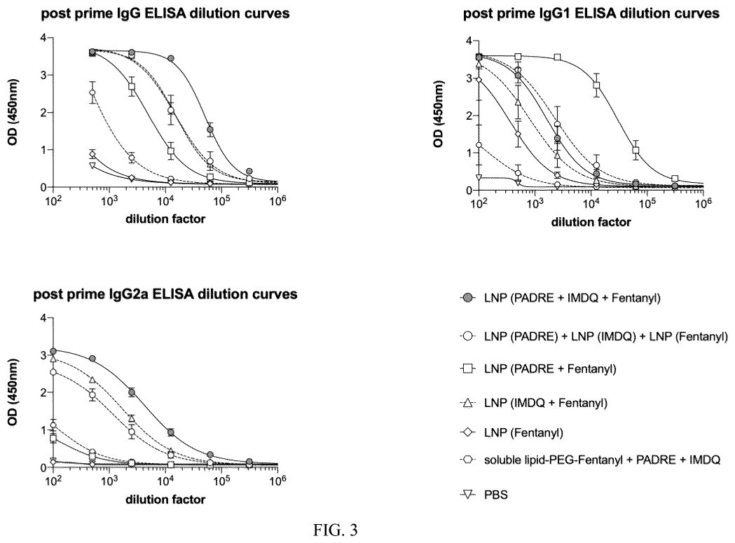

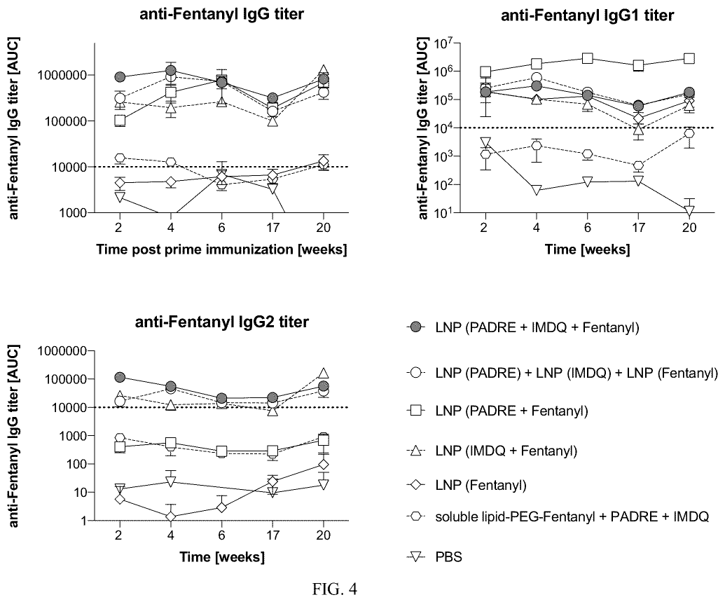

Post prime immunization sera from mice immunized with LNP(Fentanyl+PADRE+IMDQ) exhibited the highest fentanyl-specific IgG antibody titer (FIG. 3). After multiple immunizations, antibody titers in sera from immunized mice remained high, but converged (FIG. 4).

### 5.3 In Vitro Inhibition of Mu Opioid Receptor Activation

The ability of sera from immunized mice to inhibit fentanyl from binding to the mu opioid receptor was studied in vitro, using a reporter cell assay for Mu opioid receptor activation (Vandusevan et al. (2020) Biological Pharmacology 117:11310).

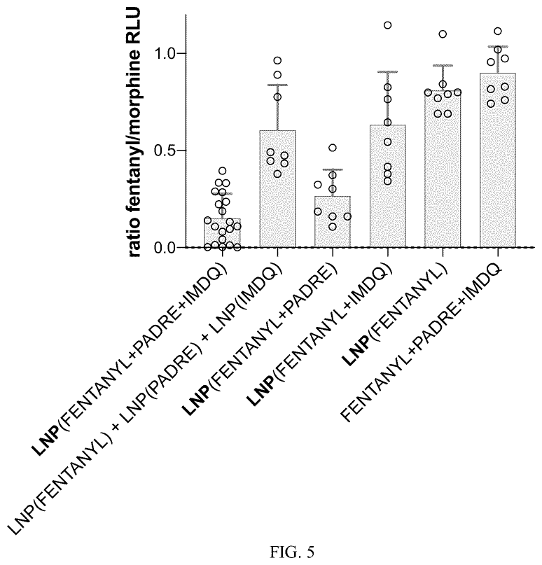

Cells were treated with either 300 nM of morphine or 10 nM of fentanyl, in presence of 2% serum of immunized mice. Luciferin substrate was added, and luminescence was recorded using a multiwell plate reader. The ratio of the luminescence recorded from fentanyl-treated cells to the luminescence recorded from morphine-treated cells was used to assess the ability of sera to sequester fentanyl, and reduce mu opioid receptor activation (FIG. 5).

Serum from mice immunized with LNP(Fentanyl+PADRE+IMDQ) was most potent in reducing mu opioid receptor activation in vitro.

### Example 6: In Vivo Neutralization Study

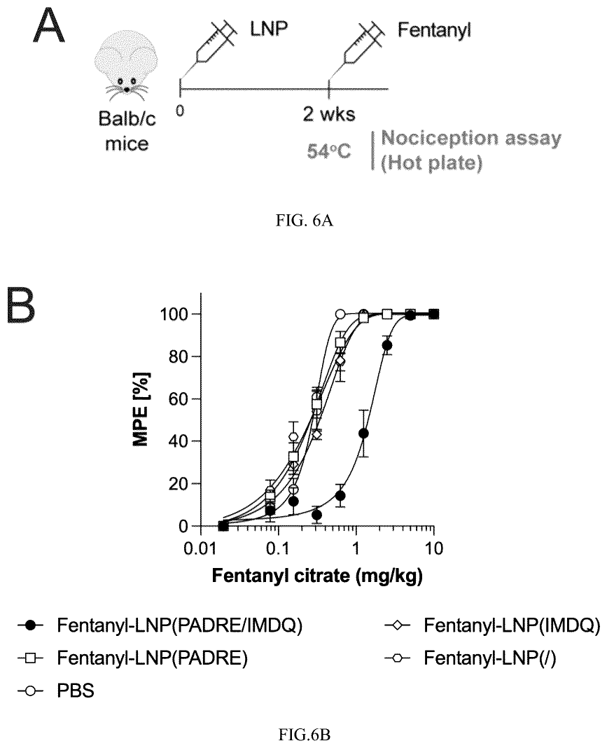

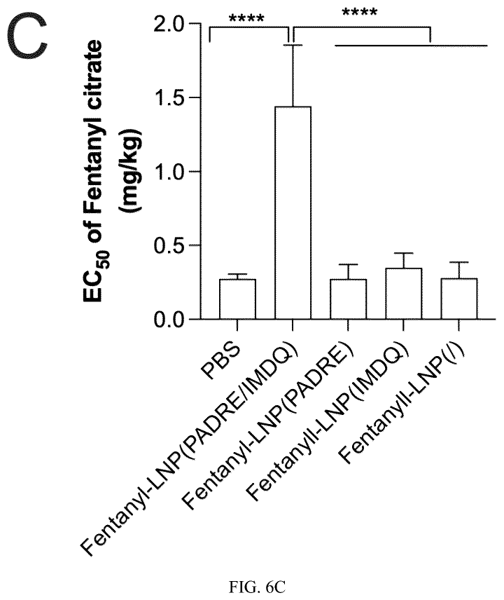

To determine the neutralization potential of the anti-fentanyl antibodies generated by the fentanyl-containing LNP formulation, it was determined whether immunized mice became protected against the antinociceptive effects of fentanyl in a hot plate assay. 2 weeks after immunization with different types of fentanyl-containing LNP formulations described in example 5 or PBS (control), mice were subjected to a hot plate assay. Briefly, mice were administered an escalating dose of fentanyl intraperitoneally, and shortly after each dose, the latency to respond to a thermal stimulus applied to the paws was measured. Specifically, the time it took for the mouse to lick/flick the hind paw or jump from the hot plate surface heated to 54° C. was measured, with a predefined maximum time of 35 seconds (FIG. 6A).

Only mice immunized with fentanyl-LNP(PADRE/IMDQ) failed to show a significant increase in resistance to the thermal stimulus upon fentanyl administration, demonstrating no latency in their response to heat, up to a fentanyl dose that was ten times higher than the dose required to elicit no nociception in non-immunized naïve mice (FIGS. 6B and 6C).

These results show that immunization with fentanyl-LNP(PADRE/IMDQ) was able to effectively neutralize fentanyl in vivo.

1. A lipid nanoparticle comprising:
a fentanyl hapten, and
a T helper peptide and/or an adjuvant,
wherein the fentanyl hapten is conjugated to the outer surface of the lipid nanoparticle, and wherein the T helper peptide and/or the adjuvant is/are encapsulated within the lipid nanoparticle.

2. The lipid nanoparticle according to claim 1, wherein the T helper peptide is a pan HLA DR-binding epitope (PADRE).

3. The lipid nanoparticle according to claim 1, wherein the T helper peptide is conjugated to an anionic peptide or polypeptide and wherein the anionic peptide or polypeptide-T helper peptide conjugate forms a complex with a cationic ionizable lipid.

4. The lipid nanoparticle according to claim 3, wherein the anionic peptide or polypeptide is an oligo-L-glutamic acid, preferably deca-L-glutamic acid.

5. The lipid nanoparticle according to claim 3, wherein the cationic ionizable lipid is selected from the group consisting of 1-(azepan-1-yl)-4,13-dioxo-5,12-dioxa-8,9-dithia-3,14-diazaheptadecane-16,17-diyl dioleate (S—Ac-7-Dog) and [(4-hydroxybutyl)azanediyl]di(hexane-6,1-diyl) bis(2-hexyldecanoate) (ALC-0315).

6. The lipid nanoparticle according to claim 1, wherein the adjuvant comprises one or more toll-like receptor (TLR) agonists, preferably a TLR 7 and 8 (TLR7/8) agonist, more preferably an imidazoquinoline (IMDQ) compound.

7. The lipid nanoparticle according to claim 1, wherein the adjuvant is conjugated to a lipid, and said lipid-adjuvant conjugate is encapsulated within the lipid nanoparticle through hydrophobic interaction.

8. The lipid nanoparticle according to claim 1, wherein the fentanyl hapten is conjugated to the outer surface of the lipid nanoparticle via a hydrophilic non-immunogenic polymer-conjugated lipid.

9. The lipid nanoparticle according to claim 8, wherein the fentanyl hapten is conjugated to the outer surface of the lipid nanoparticle via a polyethylene glycol (PEG)-modified lipid, preferably a PEGylated dialkylamine.

10. The lipid nanoparticle according to claim 1, wherein the lipid nanoparticle further comprises a structural helper lipid.

11. The lipid nanoparticle according to claim 10, wherein the structural helper lipid is a sterol, preferably cholesterol, a phospholipid, preferably 1,2-distearoyl-sn-glycero-3-phosphocholine (DSPC), or any combination thereof.

12. The lipid nanoparticle according to claim 1, comprising:
the fentanyl hapten conjugated to a PEG-modified lipid (lipid-PEG-fentanyl hapten conjugate);
a PADRE conjugated to an anionic peptide or polypeptide (anionic peptide or polypeptide-PADRE conjugate);
a cationic ionizable lipid;
a lipid-conjugated TLR agonist (lipid-TLR agonist conjugate); and
a structural helper lipid selected from the group consisting of: sterols and phospholipids, preferably a sterol and a phospholipid.

13. The lipid nanoparticle according to claim 1, wherein the lipid nanoparticle comprises:
about 0.1 mol % to about 10 mol % such as about 1.5 mol % lipid-PEG-fentanyl hapten conjugate;
about 20 mol % to about 80 mol % such as about 50 mol % cationic ionizable lipid;
about 0.1 mol % to about 10 mol % such as about 1.3 mol % lipid-TLR agonist conjugate; and
about 10 mol % to about 90 mol % such as about 47.2 mol % structural helper lipid.

14. The lipid nanoparticle according to claim 13, wherein the lipid nanoparticle comprises:
about 30 mol % to about 45 mol % such as about 38.5 mol % sterol; and
about 5 mol % to about 15 mol % such as about 8.7 mol % phospholipid.

15. The lipid nanoparticle according to claim 12, wherein the LNP has an N/P ratio between 3:1 to 30:1, preferably between 4:1 and 20:1 or between 4:1 and 10:1 such as about 5:1.

16. The lipid nanoparticle according to claim 1, wherein the lipid nanoparticle does not comprise a lipid-polymer nanoparticle.

17. The lipid nanoparticle according to claim 1, wherein the lipid nanoparticle comprises a lipid core and a lipid layer surrounding the core.

18. The lipid nanoparticle according to claim 17, wherein the lipid core comprises one or more complexes comprising a conjugate of the T helper peptide and an anionic peptide or polypeptide, preferably an anionic peptide or polypeptide-PADRE conjugate, and a cationic ionizable lipid.

19. The lipid nanoparticle according to claim 17, wherein the lipid layer comprises a conjugate of the fentanyl hapten and a hydrophilic non-immunogenic polymer-conjugated lipid, preferably a conjugate of the fentanyl hapten and a PEG-modified lipid.

20. The lipid nanoparticle according to claim 17, wherein a conjugate of the adjuvant and a lipid, preferably a lipid-TLR agonist conjugate, is present in the lipid core and/or in the lipid layer.

21. The lipid nanoparticle according to claim 1, wherein the density of the fentanyl hapten ranges from 1 to 10⁹ fentanyl hapten molecules per lipid nanoparticle.

22. The lipid nanoparticle according to claim 1, wherein the lipid nanoparticle has a mean diameter of about 10 to about 999 nm, preferably from about 10 to about 500 nm, more preferably from about 50 to about 200 nm, even more preferably from about 100 to about 150 nm.

23. A pharmaceutical composition comprising a lipid nanoparticle as defined in claim 1 and a pharmaceutically acceptable carrier.

24. The pharmaceutical composition according to claim 23, which is a vaccine composition.

25. A method for inducing an immune response against fentanyl in a subject, said method comprising administering to the subject an immunologically effective amount of a lipid nanoparticle as defined in claim 1.

26. A method for the prevention or treatment of a fentanyl abuse disorder, a fentanyl addiction or a fentanyl overdose in a subject, said method comprising administering a therapeutically effective amount of a lipid nanoparticle as defined in claim 1.

27. A method for preparing a lipid nanoparticle according to claim 1 comprising:
mixing an aqueous composition comprising the T helper peptide conjugated to an anionic peptide or polypeptide with a lipid composition comprising a cationic ionizable lipid, the fentanyl hapten conjugated to a lipid via a hydrophilic non-immunogenic polymer, preferably the fentanyl hapten conjugated to a PEG-modified lipid, a structural helper lipid, and/or a lipid-conjugated adjuvant in a suitable organic solvent, such as ethanol, to form a mixture; and
removing the organic solvent form the mixture, thereby obtaining the lipid nanoparticle.
